(19) United States
(12) Patent Application Publication
(10) Pub. No.: US 2006/0173192 A1
(43) Pub. Date: Aug. 3, 2006

(54) Aminoalkylphenyl indolone derivatives

(75) Inventors: Michael Konkel, Fair Lawn, NJ (US); Mathivanan Packiarajan, Saddle Brook, NJ (US); Heidi Chen, Fort Lee, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S

(21) Appl. No.: 11/343,989

(22) Filed: Jan. 31, 2006

Related U.S. Application Data
(60) Provisional application No. 60/649,201, filed on Feb. 2, 2005.

(51) Int. Cl. C07D 403/02

(52) U.S. Cl. 548/465; 548/482

(57) ABSTRACT

This invention is directed to aminoalkylphenyl indolone derivatives which are ligands at the GAL₃ receptor. The invention provides a pharmaceutical composition comprising a therapeutically effective amount of a compound of the invention and a pharmaceutically acceptable carrier. This invention also provides a pharmaceutical composition made by admixing a therapeutically effective amount of a compound of the invention and a pharmaceutically acceptable carrier. This invention further provides a process for making a pharmaceutical composition comprising combining a therapeutically effective amount of a compound of the invention and a pharmaceutically acceptable carrier. This invention also provides a method of treating a subject suffering from depression and/or anxiety which comprises administering to the subject an amount of a compound of the subject invention.

This application claims the benefit of U.S. Provisional Application No. 60/649,201, filed Feb. 2, 2005, the contents of which are hereby incorporated by reference.

### FIELD OF THE INVENTION

The present invention relates to compounds that are ligands at the GAL₃ receptor, and as such are useful to treat depression or anxiety.

### BACKGROUND OF THE INVENTION

Throughout this application, various publications are referenced to in full citations. The disclosures of these publications are hereby incorporated by reference into this application to describe more fully the state of the art to which this invention pertains.

Galanin is a 29-30 amino acid neuropeptide that is expressed by neurons in the brain, spinal cord and ganglia of the peripheral autonomic nervous system. Mammalian galanin is conserved between human, rat and mouse, exhibiting almost 90% amino acid homology among species, and the effects of galanin are mediated through receptors that belong in the superfamily of G protein-coupled receptors. Presently, three human galanin receptor subtypes have been cloned and characterized: GALR1 (E. Habert-Ortoli, et al., Proc. Natl. Acad. Sci., 1994, 9, 9780-9783); GALR2 (B. Borowsky, et al., Peptides, 2003, 19, 1771-1781); and GALR3 (K. E. Smith, et al., J. Biol. Chem., 1998, 273, 23321-23326).

The compounds of the present invention are ligands at the human galanin receptor subtype named “human GAL₃ receptor”. The human GAL₃ receptor, whose official gene symbol is GALR3 (see U.S. Pat. No. 6,329,197), has not been assigned an official International Union of Pharmacology (IUPHAR) nomenclature. For the purpose of clarity, the IUPHAR “provisional” name for the human GAL₃ receptor will be used throughout this application.

Data from preclinical behavioral studies, in addition to articles in the literature, evidence that targeting the galanin system is of therapeutic benefit in treating depressive and anxiety disorders. Researchers have suggested that blocking the inhibitory effects of galanin on monoamine neurotransmission with galanin receptor antagonists would be predicted to mimic or augment the action of antidepressants. In this context, central administration of galanin was found to attenuate antidepressant-induced increases in rat forebrain levels of 5-HT and noradrenaline (T. Yoshitake, et al., Neurosci. Lett., 2003, 339, 239-242).

Furthermore, it was observed that exogenous galanin alters anxiety-like behavior in rats. Research groups also observed that exogenous galanin activity in the amygdala is associated with anxiogenic-like effects under conditions of stress and high noradrenergic activity (D. A. Morilak, et al., Life Sci., 2003, 73, 715-726).

The link between the GAL₃ receptor and the effects of galanin on depression and anxiety is further evidenced from the evaluation of effects produced by selective GAL₃ small molecule ligands in behavioral models of depression or anxiety: the rat forced-swim and rat social interaction test, respectively. Administration of GAL₃ selective small molecule ligands produces a profile similar to clinically used antidepressants and anxiolytics in behavioral models of depression and anxiety (T. Blackburn, et al., PCT International Application No. PCT/US02/04608). These observations evidence that selective GAL₃ small molecule ligands are useful to treat depression and anxiety.

Current treatments for depression and anxiety are on the market. However, numerous patients do not respond to current treatments. Hence, there remains the need for alternative methods of treatment.

### SUMMARY OF THE INVENTION

The objective of the present invention is to provide compounds that are ligands at the GAL₃ receptor. The present invention relates to compounds of Formula I. 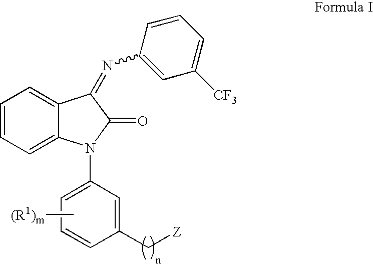
wherein R¹ is straight chained or branched C₁-C₄ alkyl, straight chained or branched C₁-C₄ alkoxy or hydroxyl;
wherein Z is —N(R²)(R³) or 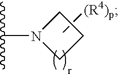
wherein R² is H or straight chained or branched C₁-C₇ alkyl, wherein the C₁-C₇ alkyl may be substituted with CN;
wherein R³ is H or straight chained or branched C₁-C₇ alkyl, wherein the C₁-C₇ alkyl may be substituted with CN;
wherein R⁴ is straight chained or branched C₁-C₄ alkyl; straight chained or branched C₁-C₄ dialkyl ether or —N(R⁵)₂;
wherein each R⁵ is independently H or straight chained or branched C₁-C₄ alkyl;
wherein m is 0 or 1;
wherein n is an integer from 1 to 5 inclusive;
wherein p is an integer from 0 to 4 inclusive; and
wherein r is an integer from 0 to 3;
or a pharmaceutically acceptable salt thereof.

In separate embodiments of the invention, the compound is selected from one of the specific compounds disclosed in the Experimental Section.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a compound of Formula I and a pharmaceutically acceptable carrier.

Moreover, the present invention provides a method of treating a subject suffering from depression comprising administering to the subject a therapeutically effective amount of a compound of Formula I. The present invention further provides a method of treating a subject suffering from anxiety comprising administering to the subject a therapeutically effective amount of a compound of Formula I.

### DETAILED DESCRIPTION OF THE INVENTION

Definitions

In the present invention, the term “straight chained or branched C₁-C₇ alkyl” refers to a saturated hydrocarbon having from one to seven carbon atoms inclusive. Examples of such substituents include, but are not limited to, methyl, ethyl, 1-propyl, 2-propyl, 1-butyl, 2-butyl, 2-methyl-2-propyl, 2-methyl-1-propyl, n-pentyl and 2-methylhexyl. Similarly, the term “straight chained or branched C₁-C₄ alkyl” refers to a saturated hydrocarbon having from one to four carbon atoms inclusive.

The term “straight chained or branched C₁-C₄ alkoxy” refers to a saturated alkoxy group having from one to seven carbon atoms inclusive with the open valency on the oxygen. Examples of such substituents include, but are not limited to, methoxy, ethoxy, n-butoxy and t-butoxy.

The term “straight chained or branched C₁-C₄ dialkyl ether” refers to two C₁-C₄ alkyl groups bonded to a central oxygen atom (i.e. [C₁-C₄ alkyl]-O-[C₁-C₄ alkyl]). The alkyl groups need not be the same and the open valency is situated as one of the alkyl groups. Examples of such substituents include, but are not limited to, dimethyl ether, diethyl ether, methyl ethyl ether and t-butyl ethyl ether.

The specific compounds disclosed in the present invention are identified by their IUPAC names. The names of the compounds were generated using the program Chemistry 4-D Draw Nomenclator™ Database (Version 7.01c, ChemInnovation Software, Inc.). According to ChemInnovation Software Inc., Nomenclator™ automatically assigns systematic names to organic structures according to IUPAC nomenclature rules. Accordingly, this application discloses the aminoalkylphenyl indolone derivatives encompassed by Formula I in accordance with IUPAC nomenclature rules.

For illustrative purposes, and without limiting the invention, the compound of example 2d has the following structure:

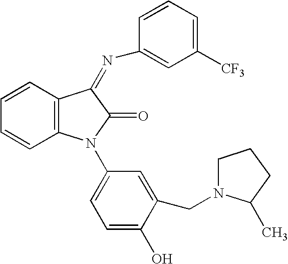

This compound is constructed from Formula I wherein m is 1; wherein R¹ is hydroxyl; wherein n is 1; wherein Z is 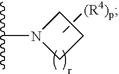
wherein r is 2; wherein p is 1; and wherein R⁴ is methyl.

Additionally, the invention further provides certain embodiments of the present invention that are described below.

In one embodiment, Z is —N(R²)(R³).

In one embodiment, R² is H or straight chained C₁-C₄ alkyl, optionally substituted with CN.

In one embodiment, R³ is H or straight chained C₁-C₄ alkyl.

In one embodiment, n is an integer from 1 to 3 inclusive.

In one embodiment, m is 0.

In one embodiment, R¹ is OH.

In one embodiment, R² is straight chained C₁-C₄ alkyl and R³ is methyl, ethyl, propyl or isopropyl.

In one embodiment, R² is straight chained C₁-C₄ alkyl and R³ is methyl, ethyl, propyl or isopropyl.

In one embodiment, Z is

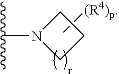

In one embodiment, r is 1 or 2.

In one embodiment, n is an integer from 1 to 3 inclusive.

In one embodiment, R⁴ is straight chained C₁-C₄ alkyl or dimethyl ether.

In one embodiment, p is 0 or 1.

Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in S. M. Berge, et al., J. Pharm. Sci. 1977, 66, 2, the contents of which are hereby incorporated by reference.

Furthermore, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, madelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, e.g., chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York 1981. Optically active compounds were also prepared from optically active starting materials.

The invention also encompasses prodrugs of the present compounds, which on administration undergo chemical conversion by metabolic processes before becoming pharmacologically active substances. In general, such prodrugs will be functional derivatives of the compounds of Formula I which are readily convertible in vivo into the required compound of Formula I. Conventional procedures for the selection and preparation of suitable prodrug derivatives are described in Design of Prodrugs, ed. H. Bundgaard, Elsevier, 1985.

Pharmaceutical Compositions

The present invention further provides a pharmaceutical composition comprising a therapeutically effective amount of a compound of Formula I and a pharmaceutically acceptable carrier. The present invention also provides a pharmaceutical composition comprising a therapeutically effective amount of one of the specific compounds disclosed in the Experimental Section and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the sex, age, weight and general condition of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

For parenteral routes such as intravenous, intrathecal, intramuscular and similar administration, typical doses are in the order of half the dose employed for oral administration.

The present invention also provides a process for making a pharmaceutical composition comprising admixing a therapeutically effective amount of a compound of Formula I and a pharmaceutically acceptable carrier. In an embodiment of the present invention the compound utilized in the aforementioned process is one of the specific compounds disclosed in the Experimental Section.

The compounds of this invention are generally utilized as the free substance or as a pharmaceutically acceptable salt thereof. One example is an acid addition salt of a compound having the utility of a free base. When a compound of Formula I contains a free base such salts are prepared in a conventional manner by treating a solution or suspension of a free base of Formula I with a molar equivalent of a pharmaceutically acceptable acid. Representative examples of suitable organic and inorganic acids are described above.

For parenteral administration, solutions of the compounds of Formula I in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of Formula I may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit.

If a liquid carrier is used, the preparation may be in the form of a syrup, emulsion, soft gelatin capsule or sterile injectable liquid such as an aqueous or non-aqueous liquid suspension or solution.

Treatment of Disorders

As mentioned above, the compounds of Formula I are ligands at the GAL₃ receptor. The present invention provides a method of treating a subject suffering from depression and/or anxiety which comprises administering to the subject a therapeutically effective amount of a compound of this invention. This invention further provides a method of treating a subject suffering from major depression and/or anxiety which comprises administering to the subject a therapeutically effective amount of a compound of this invention. In an embodiment of this invention, the subject is a human being.

The invention will be better understood from the Experimental Details which follow. However, one skilled in the art will readily appreciate that the specific methods and results discussed therein are merely illustrative of the invention as described more fully in the claims which follow thereafter. Furthermore, the variables depicted in Schemes 1-4 are consistent with the variables recited in the Summary of the Invention.

In the Experimental Section, standard acronyms are used. Examples of such acronyms include DMF (N,N-Dimethylformamide); DMSO (Dimethylsulfoxide); TEA (Triethylamine); MsCl (Methanesulfonylchloride); TsCl (Tosylsulfonylchloride); THF (Tetrahydrofuran); rt (room temperature); h (hour); and min (minutes). Furthermore, in certain instances, the methods of preparing the compounds of the invention are described generally by referring to representative reagents such as bases or solvents. The particular reagent identified is representative but is not inclusive or does not limit the invention in any way. For example, representative bases include but are not limited to K₂CO₃, TEA (Triethylamine) or DIPEA (Diisopropylethylamine).

It may be necessary to incorporate protection and deprotection strategies for substitutents such as amino, amido, carboxylic acid and hydroxyl groups in the synthetic methods described below to form the compounds of Formula I. Methods for protection and deprotection of such groups are well known in the art, and may be found in T. Green, et al., Protection Groups in Organic Synthesis, 1991, 2^{nd} Edition, John Wiley & Sons, New York.

Experimental Section

Methods of Preparing the Compounds of Formula I

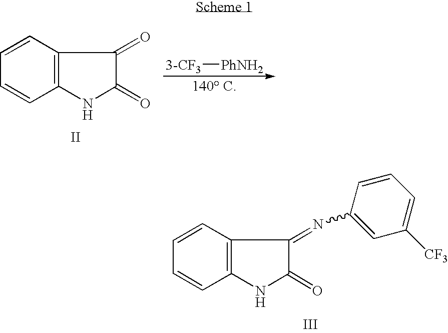

The compound of Formula III, used as starting material in Schemes 2 and 3, is synthesized from commercially available isatin and 3-(trifluoromethyl)aniline.

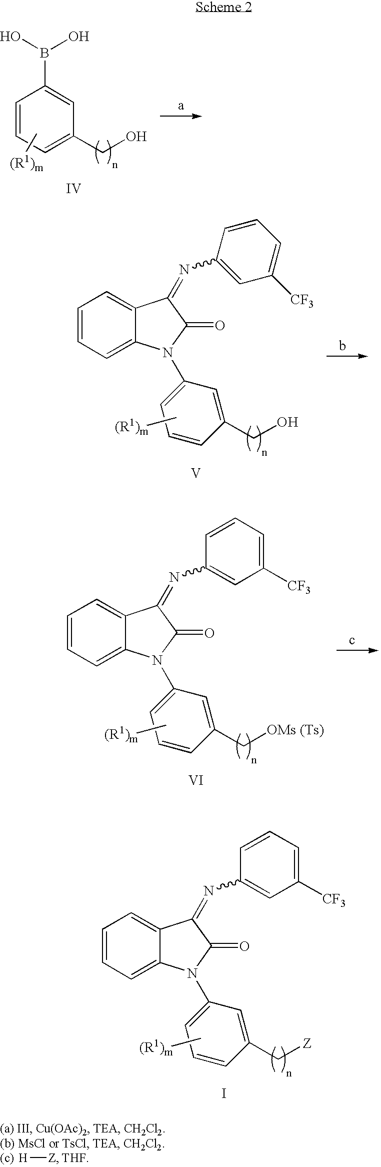

The compounds of Formula I may be synthesized according to the procedures outlined in Scheme 2 from the corresponding substituted phenylboronic acid. The compounds of Formula IV are commercially available or synthesized. First, the substituted phenylboronic acid of Formula IV is coupled with III using Cu(OAc)₂. The alcohol is further converted into the mesylate or tosylate, and the leaving group is displaced with H-Z to afford compounds of Formula I.

The intermediates of Formula IV may be prepared according to the solid-phase synthesis described in D. Hall, et al., J. Org. Chem., 2002, 67, 3-15.

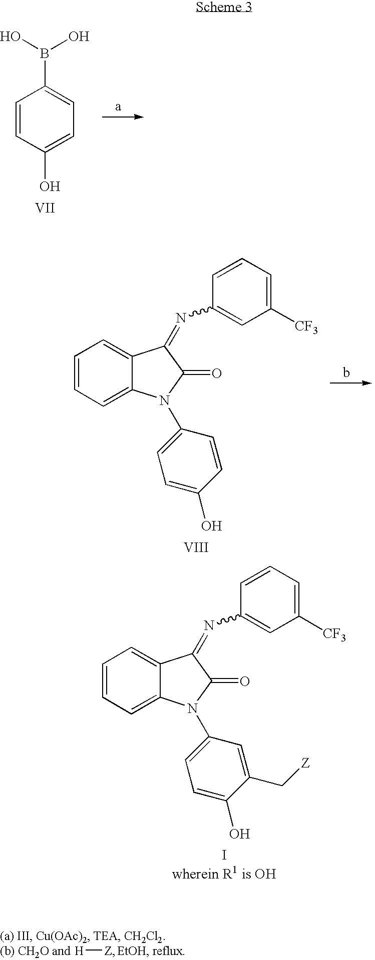

Alternatively, the compounds of Formula I, wherein R¹ is OH, may be prepared via a Mannich type condensation reaction. For example, 4-hydroxyphenylboronic acid is coupled with III using Cu(OAc)₂ to afford (3)-1-(4-hydroxyphenyl)3-{[3-trifluoromethyl}phenyl]imino-1,3-dihydro-2H-inol-2-one. An ethanolic solution of this intermediate is treated with 37% w/v aqueous formaldehyde and the appropriate amine to provide the compounds of the invention.

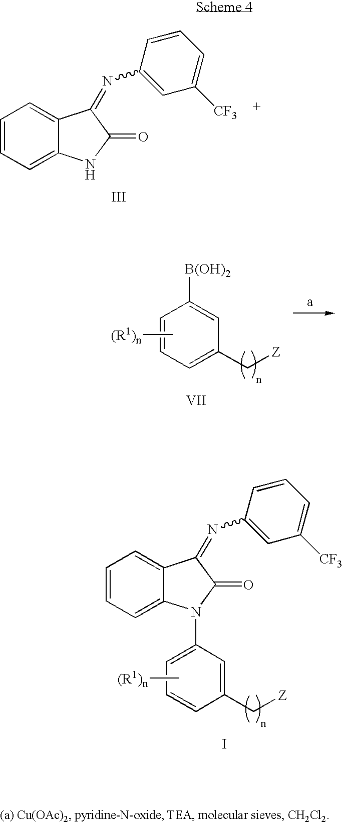

Additionally, the compounds of Formula I may be prepared as described in Scheme 4.

The intermediates of Formula VII may be prepared according to the solid-phase synthesis described in D. Hall, et al., J. Org. Chem., 2002, 67, 3-15.

General Methods: Anhydrous solvents were purchased from Aldrich Chemical Company and used as received. The NMR spectra were measured on a Bruker Avance 400 spectrometer with CDCl₃ as the solvent with tetramethylsilane as the internal standard unless otherwise noted. Chemical shifts (δ) are expressed in ppm, coupling constants (J) are expressed in Hz, and splitting patterns are described as follows: s=singlet; d=doublet; t=triplet; q=quartet; br=broad; m=multiplet; dd=doublet of doublets; dt=doublet of triplets; td=triplet of doublets; dm=doublet of multiplets. Unless otherwise noted, mass spectra were obtained using electrospray ionization (ESMS, Micromass Platform II or Quattro Micro) and (M+H)⁺ is reported. Thin-layer chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Flash column chromatography was performed on Merck silica gel 60 (230-400 mesh). Microwave-reactions were performed in a Personal Synthesizer® microwave.

Preparation of Intermediates

### 3-{[3-(Trifluoromethyl)phenyl]azamethylene}-1H-benzo[d]azolidin-2-one

Isatin (31.0 g, 0.210 mol) was combined with 3-(trifluoromethyl)aniline (132 mL, 170 g, 1.05 mol) and heated at 140° C. for 6 h. The reaction was cooled to rt, and the crystals were collected by filtration and washed with cold methanol, yielding the desired product (57.1 g, 95%). ESMS m/e: 290 (M+H)⁺.

### 1-[3-(Hydroxypropyl)phenyl]-3-{[3-(trifluoromethyl)phenyl]azamethylene}benzo[d]azolin-2-one

A mixture of 3-{[3-(trifluoromethyl)phenyl]azamethylene}-1H-benzo[d]azolin-2-one (483 mg, 1.67 mmol), Cu(OAc)₂ (675 mg, 3.67 mmol) and 3-(hydroxypropyl)phenylboronic acid (900 mg, 5 mmol) was stirred at rt overnight in CH₂Cl₂ (8 mL) with TEA (500 μL). The mixture was diluted with EtOAc (10 mL). The mixture was washed with water and brine. The organic layers were combined, dried over Na₂SO₄ and concentrated in vacuo. This intermediate product was purified by preparative TLC, eluting with 33% EtOAc in hexanes with 1% TEA (22%). ¹H NMR δ 7.54 (1H, m), 7.43 (1H, m), 7.27 (6H, m), 7.07 (1H, t, J=11.3 Hz), 6.83 (2H, m), 3.65 (2H, dt. J=7.25 Hz, 36.5 Hz), 2.77 (1H, t, J=9.5 Hz), 2.86 (1H, t, J=9.5 Hz), 1.87 (2H, dt, J=10 Hz, 46.5 Hz); ESMS m/e: 425 (M+H)⁺.

The following intermediates were prepared analogously:
1-[3-(Hydroxymethyl)phenyl]-3-{[3-(trifluoromethyl)phenyl]azamethylene}benzo[d]azolin-2-one; ESMS m/e: 397 (M+H)⁺; and
1-[4-(Hydroxy)phenyl]-3-{[3-(trifluoromethyl)phenyl]azamethylene}benzo[d]azolin-2-one; ESMS m/e: 383 (M+H)⁺.
3-[3-(2-Oxo-3-{[3-(trifluoromethyl)phenyl]azamethylene}benzo[d]azolinyl)phenyl]propyl methylsulfonate was prepared according to the procedure below:

To a cooled (−10° C.) solution of 1-[3-(hydroxypropyl)phenyl]-3-{[3-(trifluoromethyl) phenyl]azamethylene}benzo[d]azolin-2-one (129 mg, 0.303 mmol) in CH₂Cl₂ (1 mL) was added TEA (84 μL, 0.606 mmol) and MsCl (42 μL, 0.546 mmol). The reaction mixture was purged with argon and stirred at −10° C. for 1.5 h. After the reaction slowly warmed up to the rt, the mixture was poured into a separatory funnel, diluted with CH₂Cl₂, and washed with water and brine. The organic layers were combined, dried over Na₂SO₄ and concentrated in vacuo. This intermediate was used immediately without further purification. ESMS m/e: 579 (M+H)⁺.

The following compounds were prepared according to the procedures described in Scheme 2.

### EXAMPLE 1a

### 1-{3-[3-(Ethylmethylamino)propyl]phenyl}-3-{[3-(trifluoromethyl)phenyl]azamethylene}benzo[d]azolin-2-one

To a solution of 3-[3-(2-oxo-3-{[3-(trifluoromethyl)phenyl]azamethylene}benzo[d]azolinyl)phenyl]propyl methylsulfonate (25 mg, 0.043 mmol) in anhydrous THF (3 mL) was added TEA (12 μL) and N-methylethylamine (7.4 μL, 0.086 mmol). The reaction mixture was stirred at 55° C. overnight. Upon cooling to rt, the mixture was filtered through celite and THF was removed in vacuo. The final product was purified by preparative TLC and eluted with 100% EtOAc. Additional preparative TLC purification was performed and the desired product was eluted with 5% methanol in CHCl₃. (1.81 mg, 9%). ¹H NMR δ 7.79 (2H, m, J=10 Hz), 7.72-7.49 (2H, m), 7.35-7.28 (2H, m), 7.20 (2H, t, J=9.5 Hz), 7.09 (1H, d, J=9.7 Hz), 6.84-6.81 (2H, m), 6.63 (1H, d, J=8.9 Hz), 2.79 (2H, t, J=9.5 Hz), 2.62 (2H, t, J=9.5 Hz), 2.64-2.60 (2H, br), 2.47 (3H, s), 2.03-1.85 (2H, br), 1.16 (3H, m); ESMS m/e: 466 (M+H)⁺.

The following compounds were prepared analogously:

### EXAMPLE 1b

### 1-{3-[(Diethylamino)methyl]phenyl}-3-{[3-(trifluoromethyl)phenyl]azamethylene}benzo[d]azolin-2-one: ESMS m/e: 452 (M+H)⁺

### EXAMPLE 1c

### 1-{3-[(Ethylmethylamino)methyl]phenyl}-3-{[3-(trifluoromethyl)phenyl]azamethylene}benzo[d]azolin-2-one: ESMS m/e: 438 (M+H)⁺

### EXAMPLE 1d

### 3-(Methyl{[3-(2-oxo-3-{[3-(trifluoromethyl)phenyl]azamethylene}benzo[d]azolinyl)phenyl]methyl}amino)propanenitrile: ESMS m/e: 463 (M+H)⁺

### EXAMPLE 1e

### 1-{3-[3-(Diethylamino)propyl]phenyl}-3-{[3-(trifluoromethyl)phenyl]azamethylene}benzo[d]azolin-2-one: ESMS m/e: 480 (M+H)⁺

### EXAMPLE 1f

### 1-{3-[3-(Ethylpropylamino)propyl]phenyl}-3-{[3-(trifluoromethyl)phenyl]azamethylene}benzo[d]azolin-2-one: ESMS m/e: 494 (M+H)⁺

The following compounds were prepared according to the procedures described in Scheme 3.

### EXAMPLE 2a

### 1-{3-[(Diethylamino)methyl]-4-hydroxyphenyl}-3-{[3-(trifluoromethyl) phenyl]azamethylene}benzo[d]azolidin-2-one

To a stirred solution of N,N-diethylamine (28 mg, 0.39 mmol) in EtOH (2 mL) was added 37% w/v aqueous formaldehyde (40 mL, 15 mg, 0.52 mmol) and the mixture was refluxed for 30 min. An ethanolic solution of 1-[4-(hydroxy)phenyl]-3-{[3-(trifluoromethyl)phenyl]azamethylene}benzo[d]azolin-2-one (100 mg, 0.261 mmol) was added at rt and reaction was refluxed for 4 h. The solution was concentrated in vacuo and purified by preparative TLC. The titled compound was isolated as a yellow solid (63 mg, 52%). ¹H-NMR, δ 7.62-7.26 (m, 7H), 7.19 (d, 2H, J=8.8 Hz), 6.88 (d, 2H, J=8.8 Hz), 6.82 (t, 1H, J=7.6 Hz), 6.75 (d, 1H, J=7.9 Hz), 6.63 (d, 1H, J=7.2 Hz), 4.05 (dq, 4H, J=12.3 Hz, 6.2 Hz), and 1.22 (dt, 6H, J=14.1 Hz, 6.2 Hz); ESMS (m/z): 468 (M+H)⁺.

The following compounds were prepared analogously:

### EXAMPLE 2b

### 1-(3-{[(3R)-3-(Dimethylamino)pyrrolidinyl]methyl}-4-hydroxyphenyl)-3-{[3-(trifluoromethyl)phenyl]azamethylene}benzo[d]azolidin-2-one: ESMS m/e: 509 (M+H)⁺

### EXAMPLE 2c

### 1-[4-Hydroxy-3-(pyrrolidinylmethyl)phenyl]-3-{[3-(trifluoromethyl)phenyl]azamethylene}benzo[d]azolidin-2-one: ESMS m/e: 466 (M+H)⁺

### EXAMPLE 2d

### 1-{4-Hydroxy-3-[(2-methylpyrrolidinyl)methyl]phenyl}-3-{[3-(trifluoromethyl)phenyl]azamethylene}benzo[d]azolidin-2-one: ESMS m/e: 480 (M+H)⁺

### EXAMPLE 2e

### 1-(3-{[(2S)-2-(Methoxymethyl)pyrrolidinyl]methyl}-4-hydroxyphenyl)-3-{[3-(trifluoromethyl)phenyl]azamethylene}benzo[d]azolidin-2-one: ESMS m/e: 510 (M+H)⁺

### EXAMPLE 2f

### 1-{4-Hydroxy-3-[(2-methylaziridinyl)methyl]phenyl}-3-{[3-(trifluoromethyl)phenyl]azamethylene}benzo[d]azolidin-2-one: ESMS m/e: 451 (M+H)⁺

Formulations

The pharmaceutical formulations of the invention may be prepared by conventional methods in the art.

For example, tablets may be prepared by mixing the active ingredient with ordinary adjuvants and/or diluents and subsequently compressing the mixture in a conventional tabletting machine may prepare tablets. Examples of adjuvants or diluents comprise: corn starch, potato starch, talcum, magnesium stearate, gelatine, lactose, gums, and the like. Any other adjuvants or additives usually used for such purposes such as colorings, flavorings, preservatives etc. may be used provided that they are compatible with the active ingredients.

1) Tablets containing 5.0 mg of Compound 2d calculated as the free base:

| | |
|---|---|
| Compound 2d | 5.0 mg |
| Lactose | 60 mg |
| Maize starch | 30 mg |
| Hydroxypropylcellulose | 2.4 mg |
| Microcrystalline cellulose | 19.2 mg |
| Croscarmellose Sodium Type A | 2.4 mg |
| Magnesium stearate | 0.84 mg |

2) Tablets containing 0.5 mg of Compound 2d calculated as the free base:

| | |
|---|---|
| Compound 2d | 0.5 mg |
| Lactose | 46.9 mg |
| Maize starch | 23.5 mg |
| Povidone | 1.8 mg |
| Microcrystalline cellulose | 14.4 mg |
| Croscarmellose Sodium Type A | 1.8 mg |
| Magnesium stearate | 0.63 mg |

3) Syrup containing 25 mg of Compound 2d per milliliter:

| | | |
|---|---|---|
| Compound 2d | 25 | mg |
| Sorbitol | 500 | mg |
| Hydroxypropylcellulose | 15 | mg |
| Glycerol | 50 | mg |
| Methyl-paraben | 1 | mg |
| Propyl-paraben | 0.1 | mg |
| Ethanol | 0.005 | mL |
| Flavor | 0.05 | mg |
| Saccharin | 0.5 | mg |
| Water | 1 | mL |

In Vitro Methods

The pharmacological properties of the compounds of the present invention were evaluated at the cloned human GAL₃ receptor using the protocols disclosed in U.S. Pat. No. 6,329,197, the contents of which are hereby incorporated by reference.

Using this protocol, the binding by the compound to a radiolabeled ligand (¹²⁵I-labeled procine galanin) to membranes of human cloned GAL₃ receptors expressed in CHO cells was determined in vitro.

Briefly, the affinity of the compounds was measured by their ability to displace ¹²⁵I-labeled porcine galanin by incubating GAL₃ receptor expressing membranes with the compound and radioligand at 30° C. for 1 h. The binding affinities of the compounds may be determined in equilibrium competition assays, using 0.1-0.5 nM radioligand in the presence of e.g., twelve different concentrations of the displacing ligands. Incubation was terminated by rapid vacuum filtration over GF/B filters treated with 0.5% polyethyleneimine using a cell havester.

The binding affinities for the compounds in the present invention, exemplified above, at the GAL₃ receptor were determined to be 200 nM or less. For the majority of the compounds, the Ki values are 100 nM or less, and for a large group of compounds the Ki values are 25 nM or less.

What is claimed:

1. A compound having the structure: 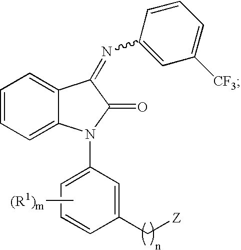
wherein R¹ is straight chained or branched C₁-C₄ alkyl, straight chained or branched C₁-C₄ alkoxy or hydroxyl;
wherein Z is —N(R²)(R³) or 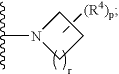
wherein R² is H or straight chained or branched C₁-C₇ alkyl, wherein the C₁-C₇ alkyl may be substituted with CN;
wherein R³ is H or straight chained or branched C₁-C₇ alkyl, wherein the C₁-C₇ alkyl may be substituted with CN;
wherein R⁴ is straight chained or branched C₁-C₄ alkyl; straight chained or branched C₁-C₄ dialkyl ether or —N(R⁵)₂;
wherein each R⁵ is independently H or straight chained or branched C₁-C₄ alkyl;
wherein m is 0 or 1;
wherein n is an integer from 1 to 5 inclusive;
wherein p is an integer from 0 to 4 inclusive; and
wherein r is an integer from 0 to 3;
or a pharmaceutically acceptable salt thereof.

2. The compound of claim 1, wherein Z is —N(R²)(R³).

3. The compound of claim 2, wherein R² is H or straight chained C₁-C₄ alkyl, optionally substituted with CN.

4. The compound of claim 3, wherein R³ is H or straight chained C₁-C₄ alkyl.

5. The compound of claim 4, wherein n is an integer from 1 to 3 inclusive.

6. The compound of claim 5, wherein m is 0.

7. The compound of claim 5, wherein R¹ is OH.

8. The compound of claim 6, wherein R² is straight chained C₁-C₄ alkyl and wherein R³ is methyl, ethyl, propyl or isopropyl.

9. The compound of claim 7, wherein R² is straight chained C₁-C₄ alkyl and wherein R³ is methyl, ethyl, propyl or isopropyl.

10. The compound of claim 1, wherein Z is

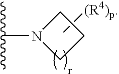

11. The compound of claim 10, wherein r is 1 or 2.

12. The compound of claim 11, wherein n is an integer from 1 to 3 inclusive.

13. The compound of claim 12, wherein R⁴ is straight chained C₁-C₄ alkyl or dimethyl ether.

14. The compound of claim 13, wherein p is 0 or 1.

15. A pharmaceutical composition comprising a therapeutically effective amount of a compound of claim 1 and a pharmaceutically acceptable carrier.

16. A process for making a pharmaceutical composition comprising admixing a therapeutically effective amount of a compound of claim 1 and a pharmaceutically acceptable carrier.

17. A method of treating a subject suffering from depression comprising administering to the subject a therapeutically effective amount of the compound of claim 1.

18. A method of treating a subject suffering from anxiety comprising administering to the subject a therapeutically effective amount of the compound of claim 1
